(19) United States
(12) Patent Application Publication
(10) Pub. No.: US 2009/0227765 A1
(43) Pub. Date: Sep. 10, 2009

(54) Monomer for making a crosslinked polymer

(75) Inventors: Carl Towns, Essex (GB); Ilaria Grizzi, Cambridge (GB); Steve O'Connor, Suffolk (GB); Annette Steudel, Cambridge (GB)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: CAMBRIDGE DISPLAY TECHNOLOGY LIMITED, Cambridgeshire (GB)

(21) Appl. No.: 11/666,025

(22) PCT Filed: Oct. 24, 2005
(86) PCT No.: PCT/GB05/04078

§ 371 Date: Mar. 18, 2008

(30) Foreign Application Priority Data
Oct. 22, 2004 (GB) 0423528.9

(51) Int. Cl. C08G 73/02; C07C 211/43; C07D 209/82; B05D 3/02

(52) U.S. Cl. 528/422; 564/433; 548/440; 427/385.5

(57) ABSTRACT

A crosslinking monomer for making a crosslinked polymer, said monomer comprising reactive leaving groups Y and Y¹ located on the same or different aryl or heteroaryl groups of the monomer and a structural unit of general formula I:

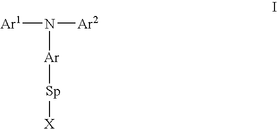

where Ar represents an aryl or heteroaryl group; Ar¹ and Ar² each independently represents a substituted or unsubstituted aryl or heteroaryl group and where any two of Ar, Ar¹ and Ar² may optionally be linked; Sp represents an optional spacer group; and X represents a crosslinkable group.

The present invention is concerned with a monomer for making a crosslinked polymer. In particular, the present invention is concerned with a monomer for making a crosslinked polymer, the crosslinked polymer being useful in an optical device.

Optical devices include organic light-emitting diodes (OLEDs), photodetectors, and photovoltaics (PVs). Such devices typically comprise one or more semiconductive polymer layers located between electrodes. Semiconductive polymers are characterised by partial or substantial pi-conjugation in the backbone or side chains.

Semiconductive polymers are now frequently used in a number of optical devices such as in light emitting diodes as disclosed in WO 90/13148; photovoltaic devices as disclosed in WO 96/16449; and photodetectors as disclosed in U.S. Pat. No. 5,523,555.

A typical LED comprises a substrate, on which is supported an anode, a cathode, and an organic electroluminescent layer, the organic electroluminescent layer being located between the anode and cathode and comprising at least one luminescent material. The luminescent material often is an electroluminescent material and further often is a polymer. In operation, holes are injected into the device through the anode and electrons are injected into the device through the cathode. The holes and electrons combine in the organic electroluminescent layer to form an exciton, which then undergoes radiative decay to give light. Other layers may be present in the LED. For example a layer of organic hole injection material, such as poly(ethylene dioxy thiophene)/polystyrene sulfonate (PEDT/PSS), may be provided between the anode and the organic electroluminescent layer to assist injection of holes from the anode to the organic electroluminescent layer.

As described in WO 96/16499, a typical photovoltaic device comprises a photoresponsive zone having first and second major surfaces and first and second electrodes provided on respective ones of the first and second major surfaces of the photoresponsive zone. The photoresponsive zone comprises an electron accepting polymer and an electron donating polymer which may be provided as separate layers or as a blend. Under short-circuit conditions, an internal electric field exists within the photoresponsive zone. The orientation of the internal electric field is such that electrons migrate to and are collected at the contact with the lowest work function, generally an aluminium, magnesium or calcium electrode while holes move towards the electrode with the higher work function, such as an indium tin oxide electrode. Thus, a photocurrent is generated and may be used, for example, to provide electrical power as in the case of a solar cell, for example, or to enable detection of part of a light pattern such as an image for use in an image sensor.

Semiconductive polymers can exhibit a wide range of photophysical properties (such as the π-π* bandgap and photoluminescent yield); optical properties (such as refractive index and its dispersion); electronic properties (such as hole- and electron-transport energy levels, and hole- and electron-mobilities); and processing properties (such as solvent solubility, phase transition temperature, crystallinity and phase-transition temperatures). These properties are largely controlled by the chemical structure of the polymer. In this regard, these properties largely may be controlled within a range by appropriate selection of the backbone units and side chains of the polymer.

When a polymer is used in an optical device, the polymer preferably is soluble in common organic solvents to facilitate its deposition during device manufacture. One of the key advantages of this solubility is that a polymer layer can be fabricated by solution processing, for example by spin-casting, ink-jet printing, screen-printing, dip-coating etc.

In certain devices it can be desirable to cast multiple layers, i.e., laminates, of different materials on a single substrate surface. For example, this could be to achieve optimisation of separate functions, for example electron or hole charge transport, luminescence control, photon-confinement, exciton-confinement, photo-induced charge generation, and charge blocking or storage.

In this regard, it can be useful to be able to fabricate multilayers of polymers to control the electrical and optical properties, for example, across the polymer stack. This can be useful for optimum device performance. Optimum device performance can be achieved, for example, by careful design of the electron and hole transport level offset, of the optical refractive index mismatch, and of the energy gap mismatch across an interface. Such heterostructures can, for example, facilitate the injection of one carrier but block leakage of the opposite carrier and/or prevent exciton diffusion to the quenching interface. Thereby, such heterostructures can provide useful carrier and photon confinement effects.

It also can be useful to be able to fabricate multilayers in order to provide a protective layer in the device structure. In this regard, taking one example, it is believed that PEDT/PSS may have a deleterious effect on the electroluminescent layer of polymer OLEDs (P-OLEDs). Without wishing to be bound by theory, it is thought that this may be due to electrochemical reactions between the PEDT:PSS layer and the electroluminescent layer (i.e. the layer in which holes and electrons combine to form an exciton). It is thought that this results in quenching of luminescence and progressive increase in required drive voltage. Accordingly, it may be desirable to provide a protective layer between PEDT:PSS and the electroluminescent layer.

However, preparation of polymer laminates is not trivial. In particular, the solubility of initially cast or deposited layers in the solvents used for succeeding layers can be problematic. This is because solution deposition of the subsequent polymer layer can dissolve and destroy the integrity of the previous layer.

One option for overcoming this problem is to work with precursor polymer systems. Precursor systems of PPV (polyphenylene vinylene) and PTV (polythienylene vinylene) are known in this art.

However, it is clearly undesirable to restrict the polymer in a polymer device to that class of polymers that may be formed from soluble precursor polymers. Furthermore, the chemical conversion process required for precursor polymers involves extreme processing conditions and reactive by-products that may harm the performance of the prior layers in the finished device.

A further option is to use a luminescent film-forming solvent processable polymer which is crosslinked, as disclosed in WO96/20253. It is stated in WO96/20253 that because the thin films resist dissolution in common solvents this enables deposition of further layers, thereby facilitating device manufacture. The use of azide groups attached to the polymer main chain is mentioned as an example of thermal crosslinking.

U.S. Pat. No. 6,107,452 discloses a method of forming a multilayer device wherein oligomers comprising terminal vinyl groups are deposited from solution and cross-linked to form insoluble polymers onto which additional layers may be deposited.

Muller et al., Nature, Volume 421, 20 Feb. 2003 pages 829-832 demonstrates a three colour organic light-emitting device by solution processing using emitter polymers with photoresist properties; that is, soluble polymers, which can be cured photochemically to yield an insoluble form (polymer network). Crosslinking is achieved by incorporating oxetane-functionalised spirobifluorene repeat units in the polymer. The crosslinked polymers are described only for use as emissive materials in a light-emitting device.

Faber et al., J. Macro. Mol. SCI.—Pure Appl. Chem., A38(4), 353-364 (2001) reports blue electroluminescence crosslinkable polymers containing fluorene/phenylene alternating repeat units in the main chain and oxetane side chains. To form the polymers, the Suzuki polycondensation method is applied to polymerise oxetane functionalised phenylene comonomers with fluorene comonomers. The focus of this document is on polymers for blue light emission in a light-emitting device.

Braig et al, Macromol. Rapid Commun. 21, 583-589, 2000 discloses polymerisation of N,N′-diphenylbenzidine monomer with an oxetane functionalised monomer to form a cross-linkable polymer. These polymers have saturated segments along the polymer backbone.

Klarner et al., Chem. Mater. 1999, 11, 1800-1805 discloses a series of crosslinkable oligo and poly(dialkylfluorenes). The styryl-functionalised oligomers and polymers can be crosslinked by the vinyl end groups. Scheme 1 shows Yamamoto type polymerisation of 2,7-dibromo-9,9-di-n-hexylfluorene in the presence of crosslinking end caps. Scheme 2 shows a fluorene copolymer containing internal crosslinking sites in the 9,9 position of some of the fluorene units. It is said that the polymers produced by scheme 1 enable fabrication of colour-fast blue light-emitting devices.

Multilayer devices comprising a cross-linked hole transporting layer are known. Kim et al, Synthetic Metals 122 (2001), 363-368 discloses polymers comprising triarylamine groups and ethynyl groups which may be cross-linked following deposition of the polymer. The ethynyl groups are present in the polymer main chain. This will result in a change in functionality of the polymer as compared with the corresponding polymer with no crosslinking ethynyl groups. In particular, the presence of the crosslinking ethynyl groups will interfere in a negative sense with the hole transporting properties of the polymer. Additionally the thermal crosslinking of ethynyl groups is demanding requiring temperatures greater than 150 C. Even at these temperatures one is not likely to achieve full conversion of all the ethynyl groups. Further the unreacted ethynyl groups are likely to be deleterious to device performance since the ethynyl groups could undergo oxidation and the terminal proton is weakly acidic. Also the crosslinking chemical products derived from ethynyl functionalities are complex involving the formation of vinyl groups, which may be source of chemical instability and hence undesirable in terms of their effect on device performance

J. Appl. Phys. 2003, 94(5), 3061-3068 discloses a hole transporting layer of a poly(triphenylamine) with cross-linkable styryl end-capping groups. As a result, there are only two cross-linkable groups per polymer which may not be sufficient to render the polymer insoluble.

Chem. Mater. 2003, 15(7), 1491-1496 discloses a series of copolymers made by copolymerisation of substituted bis(diarylamino)biphenyl acrylate monomers and cinnamate acrylate monomers. Cross-linking of the polymers is achieved through polymerisation of the cinnamate group. The copolymers have saturated and therefore non-conjugated backbones, which detracts from device performance.

It will be understood from the above that there have been reports of crosslinkable polymeric hole transport and/or electroluminescent materials to facilitate the construction of solution processed multilayer devices. In the case of cross-linkable hole transporting materials, it will further be understood that the crosslinking groups are provided as end-capping groups or as repeat units of a non-conjugated polymer.

In the case of introducing crosslinking groups as side groups along the polymer chain, this typically is achieved in the aforementioned prior art by using an arylene comonomer, such as a fluorene comonomer, which is functionalised with the crosslinking group. In the case of hole transport materials the present inventors have recognised that a necessity to use functionalised fluorene units effectively limits the extent to which the hole transport properties of the material can be tuned and therefore optimised. This is because fluorene is an electron transporting group and its presence in a hole transport material may or may not be desirable depending on the device structure in which optimal hole transport properties are desired.

Moreover, importantly, the present inventors have found that such functionalised fluorenes, in any case, do not crosslink very efficiently. This means that in practice the polymer may remain partially soluble even after crosslinking. Consequently, the integrity of the layer may not be preserved when a subsequent layer is deposited thereon.

Therefore, although these functionalised fluorenes offer advantages in being able to introduce a selected number of crosslinking groups spaced along a conjugated polymer backbone, there are significant disadvantages with respect to efficiency and, in some cases, functionality.

On this basis, the present inventors formulated the technical problem to be solved by the present invention which was to provide new means for incorporating crosslinking groups into a polymer, and particular into a hole transporting polymer, preferably so that the efficiency of crosslinking of the crosslinking groups is improved as compared with functionalised fluorene units.

Thus, it was an aim of the present invention to provide a new crosslinking monomer for making a crosslinked polymer.

The present inventors have at least partially solved the technical problem by providing, in a first aspect of the invention, an optionally substituted crosslinking monomer for making a crosslinked polymer, said monomer comprising reactive leaving groups Y and Y¹ located on the same or different aryl or heteroaryl groups of the monomer and a structural unit of general formula I:

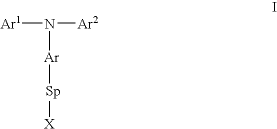

where Ar represents an aryl or heteroaryl group; Ar¹ and Ar² each independently represents a substituted or unsubstituted aryl or heteroaryl group and where any two of Ar, Ar¹ and Ar² may optionally be linked; Sp represents an optional spacer group; and X represents a crosslinkable group. It will be understood that crosslinkable group X typically is a terminal group, more preferably in the case where X comprises a double bond the crosslinkable group X terminates in a ═CH2 group.

It will further be understood that typically X is located such that it is pendant from the polymer backbone when the monomer is polymerised, prior to crosslinking. Typically Ar¹ and Ar² are located such that when the monomer is polymerised, Ar¹ and Ar² are incorporated into the polymer backbone.

When -(—Ar—Sp-X is pendant from the polymer backbone, in one embodiment, it is preferred that -(—Ar—Sp-X does not represent a benzocyclobutanyl group or a substituted C₆₋₁₂ arylene group containing one or more substituents selected from the group consisting of benzocyclobutane, azide, oxirane, di(hydrocarbyl)amino, cyanate ester, hydroxy, glycidyl ether, C₁₋₁₀ alkylacrylate, C₁₋₁₀ alkylmethacrylate, ethenyl, ethenyloxy, perfluorethenyloxy, ethynyl, maleimide, nadimide, tri(C₁₋₄)-alkylsiloxy, tri(C₁₋₄) alkylsilyl and halogenated derivatives thereof.

The monomer according to the first aspect of the present invention is suitable for making a conjugated polymer. Reactive leading groups Y and Y¹ leave during polymerisation. Thus the aryl or heteroaryl group(s) on which Y and Y¹ are located in the monomer can be conjugatively linked to an adjacent repeat unit in the polymer backbone by a direct bond to a carbon-carbon double bond, aryl group or heteroaryl group in the adjacent repeat unit. In this way a conjugated polymer can be formed.

By virtue of Y and Y¹ being located on aryl or heteroaryl groups, the backbone of a polymer made from the monomer will contain the aryl or heteroaryl groups on which Y and Y¹ were located.

It will be understood that following polymerisation of the monomer, the residue of the monomer that is incorporated into the polymer as a repeat unit will comprise general formula I.

The monomer according to the present invention is advantageous because the crosslinkable groups undergo efficient crosslinking. In particular, the crosslinking group(s) in the monomer according to the present invention has been found to undergo more efficient crosslinking than those in a monomer containing a fluorene group functionalised with crosslinking groups. Moreover, the monomer according to the invention combines hole transport functionality and crosslinking functionality in a single monomer. This means that the hole transporting functionality of the polymer is not affected in a negative sense by incorporation of the present monomer. Therefore, the hole transport properties of the polymer can be tuned over a wider range.

The monomer will contain two or more reactive leaving groups, which participate in polymerisation. Preferably, the monomer contains two reactive leaving groups (Y and Y¹). Further preferably, at least one reactive group (Y and/or Y¹) is located directly on Ar, Ar¹ or Ar². However, this is not essential as will be seen from the fifth embodiment described below.

Preferably, Y and Y¹ each independently is a leaving group capable of undergoing a metal mediated cross-coupling reaction.

More preferably, Y and Y¹ independently are selected from the group consisting of boronic acid, boronic acid ester (preferably C1-C6), borane (preferably C1-C6), halide or triflate.

Most preferably, Y and Y¹ independently are selected from the group consisting of bromine and boronic acid ester.

Y and Y¹ may be the same or different.

Where one of Ar, Ar¹ and Ar² is linked to another one of Ar, Ar¹ and Ar², the link preferably is a direct bond.

X preferably represents a styrene group. Other possible x groups include acetylene, azide, oxetane and groups comprising a 4 membered ring, such as a C4 ring, fused to an aromatic or heteroaromatic group, for example phenyl as in benzocyclobutane. In the embodiment where Sp is not present X may be fused to Ar, for example X may represent a 4 membered ring such as a C4 ring fused to Ar, preferably phenyl.

Preferably, Ar¹ and/or Ar² represents a substituted or unsubstituted phenyl group. Other possible Ar¹ and/or Ar² groups include substituted phenyl; substituted or unsubstituted fluorene; substituted or unsubstituted biphenyl; substituted or unsubstituted thiophene; and substituted or unsubstituted pyridine.

The optional substituents of the monomer include further crosslinkable groups. However it is preferred that the monomer comprises only one crosslinkable group.

The spacer group (Sp), where present can be any suitable group, such as an n-alkyl or branched alkyl or alkoxy group. Further, the spacer group can be a polyethylene glycol chain of any suitable length i.e. —(CH₂CH₂O)ₙ—, where n is an integer. Still further, the spacer group can be a phenyl or substituted phenyl or phenoxy group. One preferred spacer group is an aromatic group for improving the efficiency of crosslinking. Aromatic spacer groups are particularly preferred for use with unsaturated crosslinkable groups, for example vinyl groups, that are capable of conjugating with the aromatic spacer group. Another preferred spacer group is n-alkyl or branched alkyl. The presence of a flexible spacer, in particular these alkyl groups, allows the crosslinkable group X to orientate itself into an optimal crosslinking conformation. The flexible spacer may be used with both saturated and unsaturated crosslinkable groups X. One particularly preferred crosslinkable group X for use with flexible spacers is benzocyclobutane.

For ease of synthesis, it is preferred that Ar represents an unsubstituted phenyl group. Other possible Ar groups include substituted phenyl; substituted or unsubstituted fluorene; substituted or unsubstituted biphenyl; substituted or unsubstituted thiophene; and substituted or unsubstituted pyridine.

Where Ar is phenyl, preferably X is provided in the para-position of the Ar.

In a first preferred embodiment of the first aspect, the monomer comprises general formula II:

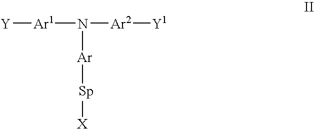

where y, y, Ar, Ar¹, Ar² Sp and X are as defined above.

In general formula II, for ease of synthesis, it is preferred that Ar represents an unsubstituted phenyl group. Other possible Ar groups include substituted phenyl; substituted or unsubstituted fluorene; substituted or unsubstituted biphenyl; substituted or unsubstituted thiophene; and substituted or unsubstituted pyridine.

Where Ar is phenyl, preferably X is provided in the para-position of the Ar.

More preferably, the monomer comprises general formula III:

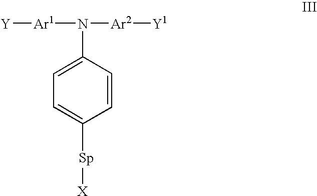

where Ar¹, Ar², Y, Y¹, Sp, and X are as defined above.

Preferred crosslinking monomers according to the first embodiment of the first aspect comprise general formula IV or general formula V:

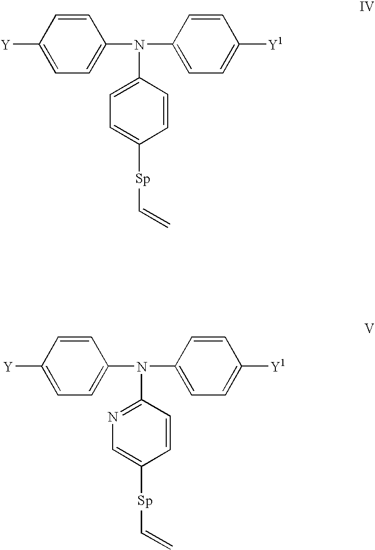

where Sp, Y and Y¹ are as defined above.

Further preferred monomers according to the first embodiment of the first aspect comprise general formula VI or general formula VII:

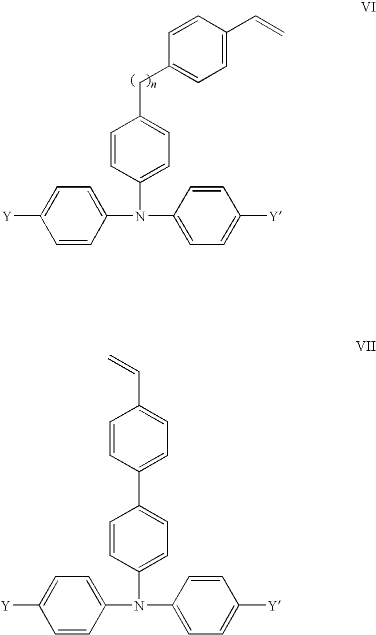

where Y and Y¹ are as defined above.

In a second preferred embodiment of the first aspect, the monomer comprises general formula VIII:

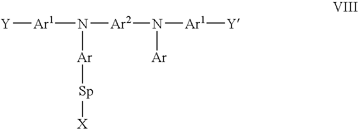

where Ar, Ar¹, Ar², Sp, X, Y and Y¹ are as defined anywhere above.

The monomer according to the second preferred embodiment may contain a second crosslinkable group (X).

A preferred monomer according to the second embodiment of the first aspect comprises general formula IX:

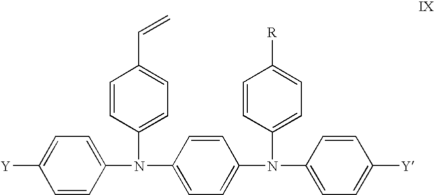

where Y and Y¹ are as defined above and R represents a substituent group, such as optionally substituted alkyl, alkoxy, alkylthiol, aryl or heteroaryl, preferably alkyl or alkylphenyl.

In a third preferred embodiment of the first aspect, the monomer comprises general formula XVIII:

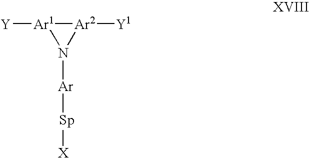

where Ar, Ar¹, Ar², Sp, X, Y and Y¹ are as defined anywhere above.

A preferred monomer according to the third embodiment comprises general formula XI or XII:

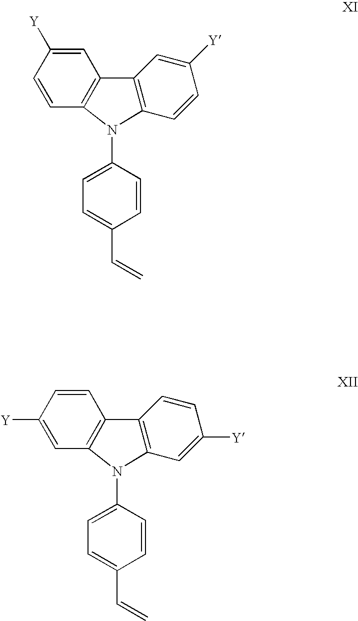

where Y and Y¹ are as defined above.

In a fourth preferred embodiment of the first aspect, the monomer comprises general formula XIII:

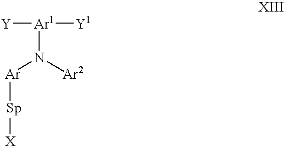

where Ar, Ar¹, Ar², Sp, X, Y and Y¹ are as defined anywhere above.

A preferred monomer according to the fourth embodiment comprises general formula XIV or general formula XV:

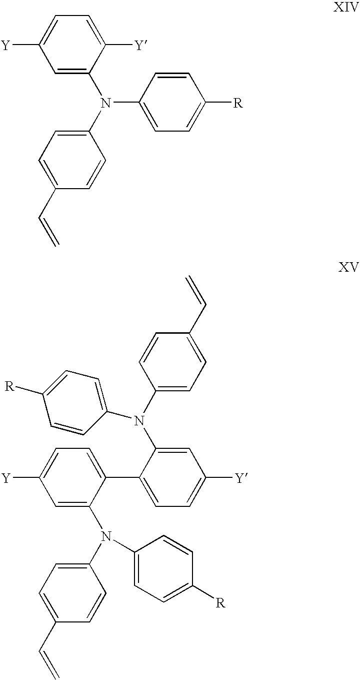

where Y and Y¹ are as defined above and R represents a substituent group such as optionally substituted alkyl, alkoxy, alkylthiol, aryl or heteroaryl, preferably alkyl or alkylphenyl. Each R may be the same or different.

In the first, second, third and fourth embodiments, the monomer comprising general formula I is capable of being conjugatively linked to the polymer backbone because the reactive groups Y and Y¹ are located directly on Ar¹ and/or Ar².

In a fifth preferred embodiment of the first aspect of the present invention, the monomer comprises general formula XVI:

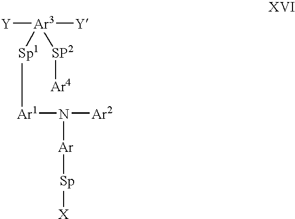

where Ar, Ar¹, Ar², Sp, y, Y¹ and X are as defined anywhere above; Sp¹ and Sp² each independently represents an optional spacer group; Ar³ represents an aryl or heteroaryl group and Ar⁴ represents an aryl or heteroaryl group.

According to the fifth embodiment, general formula I can be either conjugatively or non-conjugatively linked to the polymer backbone. Where there is an interruption in conjugation between Ar¹ and Ar³, general formula I will be non-conjugatively linked to the polymer backbone. Where there is no interruption in conjugation between Ar¹ and Ar³, general formula I will be conjugatively linked to the polymer backbone.

A preferred monomer according to the fifth embodiment comprises general formula XVII:

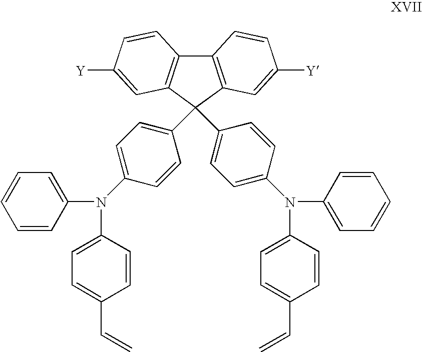

where Y and Y¹ are as defined above.

Preferred groups Ar, Ar¹, Ar², Y, Y¹, Sp, and X of the first, second, third and fourth and fifth embodiments of the first aspect are as described anywhere above.

A second aspect of the invention provides the use of a crosslinking monomer as defined in relation to the first aspect, in a method for producing a crosslinked polymer.

A third aspect of the invention provides a method for producing a crosslinkable polymer comprising the steps of:
(i) providing a plurality of monomers containing at least 1 mol % of crosslinking monomers as defined in relation to the first aspect; and
(ii) polymerising the plurality of monomers to form a polymer.

In step (i), the plurality of monomers preferably contains from 1 to 25 mol % more preferably from 5 to 25 mol % of monomers defined in relation to the first aspect of the invention. More preferably, the plurality of monomers contains from 1 to 15 mol %, still more preferably from 1 to 10 mol % or 5 to 15 mol %, even more preferably from 5 to 10 mol % of monomers defined in relation to the first aspect. The concentration of monomers defined in relation to the first aspect the plurality of monomers should be selected according to the degree of crosslinking desired in the final crosslinked polymer.

Generally the crosslinking step (iii) occurs after the polymer formed in step (ii) has been deposited as a film onto a substrate. The polymer containing the crosslinkable group typically is prepared in step (ii) as a linear chain extended solution soluble polymer which can be deposited onto a substrate by solution processing. Crosslinking during step (ii) generally is undesirable as this would result in a three dimensional (crosslinked) intractable or gel like material that could not then be manipulated into a device as a thin film.

In the method according to the third aspect, preferably the plurality of monomers are provided in solution in step (i).

Further, in the method according to the third aspect, the plurality of monomers preferably further contains a plurality of non-crosslinking monomers, each containing an aryl or heteroaryl group such that each residue of a non-crosslinking monomer that is incorporated into the polymer as a repeat unit comprises the aryl or heteroaryl group.

Suitable aryl and heteroaryl groups (to be present in co-repeat units) include:
optionally substituted hydrocarbyl aryl repeat units, in particular fluorene (particularly 2,7-linked fluorene), spirofluorene, indenofluorene and phenyl
optionally substituted heteroaryl groups
optionally substituted triarylamines or carbazoles.

The fluorene group preferably is 9,9-substituted fluorene. One preferred fluorene group is optionally substituted 9,9-diarylfluorene. Another preferred fluorene group is optionally substituted 9,9-dialkylfluorene, more preferably dioctylfluorene. Since the crosslinking function is provided by the repeat units derived from the monomer according to the first aspect of the invention, substitution positions on the fluorene group are available for control of the electronic and physical properties of the polymer (e.g. solubility, glass transition temperature and electron affinity).

In the method according to the third aspect, the plurality of non-crosslinking monomers may comprise two or more different types of non-crosslinking monomers. For example, the plurality of non-crosslinking monomers may comprise a first non-crosslinking monomer containing a carbazole group and a second non-crosslinking monomer that is different from the first monomer. The second non-crosslinking monomer may, for example, contain a fluorene group. When the plurality of non-crosslinking monomers comprises a first non-crosslinking monomer containing a carbazole group and a second monomer containing a fluorene group, the plurality of crosslinking monomers preferably comprises monomers according to the third embodiment of the first aspect of the present invention.

Alternatively, in the method according to the third aspect, the plurality of non-crosslinking monomers may consist of one type of non-crosslinking monomer, as described anywhere herein.

In one embodiment of the third aspect, the plurality of monomers further contains emitting monomers and/or hole transporting monomers, which are different from the crosslinking monomers. Emitting monomers contain emissive units, such as units 7 to 21 illustrated below, and hole transporting monomers contain hole transport units, such as a triarylamine group or a carbazole group.

Preferred carbazole-containing monomers comprise general formula 22:

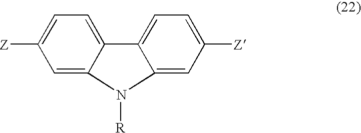

Where Z comprises a reactive leaving group Y and Z′ comprises a reactive leaving group Y¹. Y and Y¹ independently may be as defined anywhere herein. R represents hydrogen or a substituent group.

Preferred substituted carbazole groups are substituted by R′ as shown in formula 23:

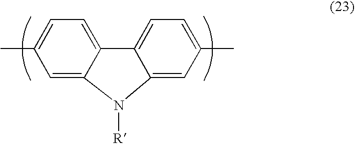

Suitable R′ substituent groups include alkyl, aryl, and heteroaryl.

Preferred triarylamine repeat units are selected from repeat units of formulae 1 to 6:

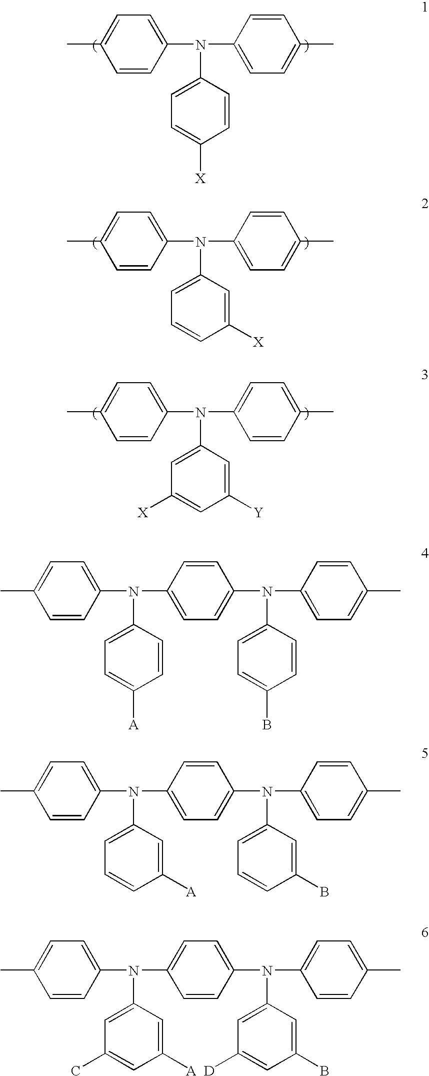

wherein A, B, C and D are independently selected from H or a substituent group. Preferred substituent groups include optionally substituted, branched or linear alkyl, aryl, perfluoroalkyl, thioalkyl, cyano, alkoxy, heteroaryl, alkylaryl and arylalkyl groups. Most preferably, each of A, B, C and D represents a C₁₋₁₀ alkyl group. The repeat unit of formula I is most preferred.

Particularly preferred heteroaryl co-repeat units include units of formulae 7 to 21:

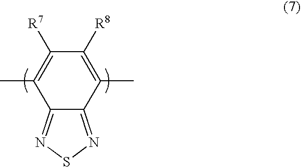

wherein R₆ and R₇ are the same or different and are each independently hydrogen or a substituent group, preferably alkyl, aryl, perfluoroalkyl, thioalkyl, cyano, alkoxy, heteroaryl, alkylaryl or arylalkyl. For ease of manufacture, R₆ and R₇ are preferably the same. More preferably, they are the same and are each a phenyl group.

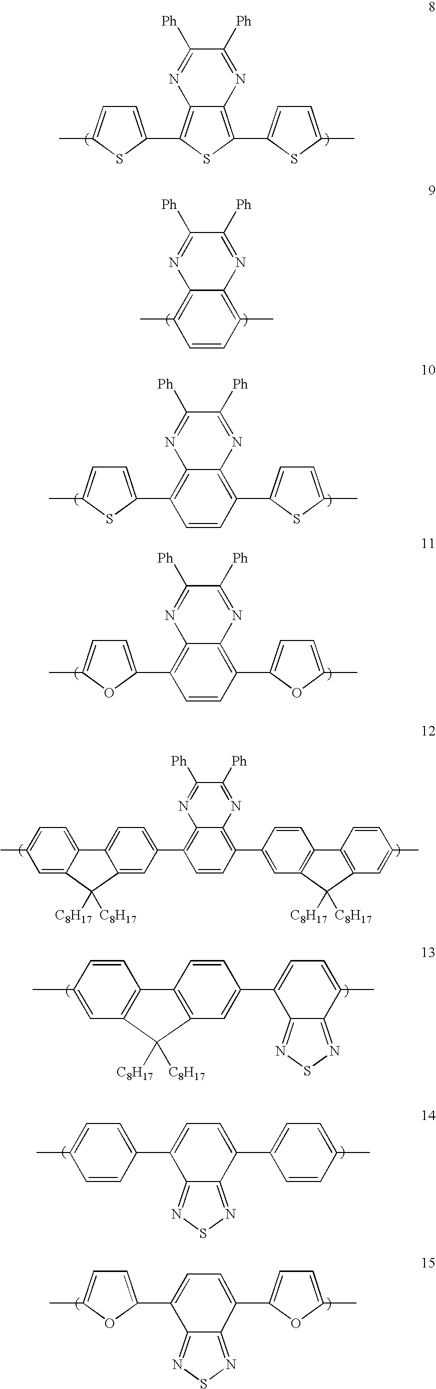

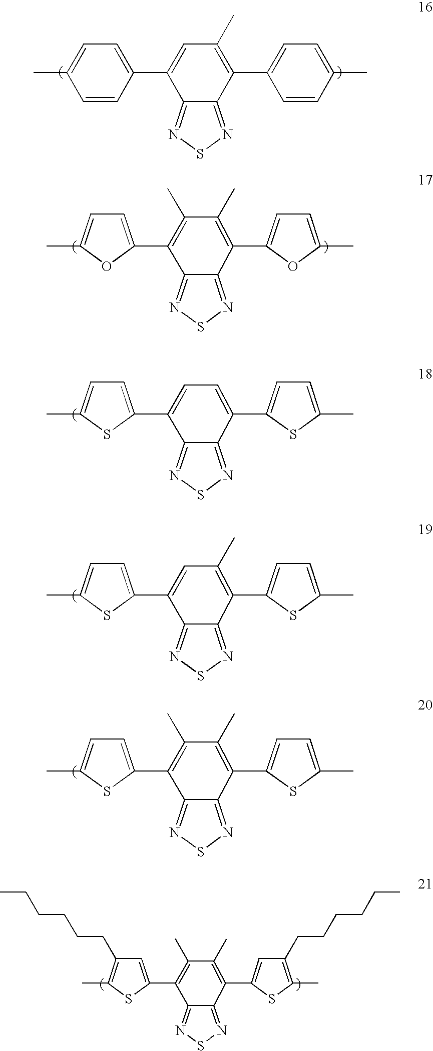

Further suitable arylene repeat units are known in this art, for example as disclosed in WO 00/55927 and WO 00/46321, the contents of which are incorporated herein by reference.

Where the plurality of monomers further contains a plurality of non-crosslinking monomers, each containing a fluorene group, the plurality of monomers preferably contains from 1 to 25 mol %, more preferably from 5 to 25 mol % or 1 to 15 mol %, still more preferably from 1 to 10 mol % or 5 to 15 mol %, even more preferably from 5 to 10 mol % of monomers defined in relation to the first aspect of the invention.

In another embodiment of the third aspect, the plurality of monomers consists of monomers as defined in relation the first aspect.

Referring to step (ii), polymerisation suitably is carried out by a metal mediated cross-coupling reaction, such as Suzuki polymerisation as disclosed in WO 00/53656 for example, or Yamamoto polymerisation as disclosed in, for example, “Macromolecules”, 31, 1099-1103 (1998). Suzuki polymerisation entails the coupling of halide and boron derivative functional groups; Yamamoto polymerisation entails the coupling of halide functional groups. Accordingly, it is preferred that each monomer is provided with two reactive functional groups wherein each functional group is independently selected from the group consisting of (a) boron derivative groups selected from boronic acid groups, boronic ester groups and borane groups; (b) halide groups; and (c) triflate groups.

The method according to the third aspect, may include a further step (iii) of crosslinking the polymer from step (ii) to form a crosslinked polymer. Step (iii) typically comprises heating the polymer to perform crosslinking. As such, a preferred crosslinking technique is thermal crosslinking.

Any suitable temperature and heating time may be used during thermal crosslinking. A heating temperature of greater than 70° C. is typical. Generally, a suitable temperature will be in the range of from about 180° C. to about 200° C., with higher temperatures in this range being preferred. Using the conditions of heating at 200° C. for 1 hour provided for the fluorene-containing polymers exemplified below, the skilled person will be able to determine an appropriate heating temperature and time to enable crosslinking of other polymers.

Another possible crosslinking technique is UV crosslinking. In the method according to the third aspect, it is preferred that each aryl or heteroaryl group on which a Y and/or Y¹ reactive leaving group is located in the plurality of crosslinking monomers is conjugatively linked by a direct bond to a carbon-carbon double bond, aryl group or heteroaryl group in another monomer during polymerising in step (b).

It is further preferred that the method according to the third aspect is a method for producing a crosslinked conjugated polymer.

A fourth aspect of the present invention provides a crosslinked polymer preparable by the method as defined in relation to the third aspect. Preferred features of the polymer are as discussed above in relation to the first, second and third aspects.

The crosslinked polymer preferably has a HOMO level of less than or equal to −5.5 eV, more preferably around −4.8 to −5.5 eV.

Typically, the crosslinked polymer will be semiconducting.

Preferably, the crosslinked polymer comprises a conjugated polymer.

A preferred hole transport polymer comprises a carbazole repeat unit and a repeat unit corresponding to the residue of a monomer disclosed in relation to the first aspect of the present invention. This preferred polymer may further contain a fluorene repeat unit, preferably at a ratio of 1 fluorene repeat unit:2 carbazole repeat units. The residue of a monomer disclosed in relation to the first aspect of the present invention preferably is present in the polymer at a concentration of from 1 to 25 mol %, more preferably from 5 to 25 mol % or 1 to 15 mol %, still more preferably from 1 to 10 mol % or 5 to 15 mol %, even more preferably from 5 to 10 mol %.

A fifth aspect of the present invention provides the use of a crosslinked polymer as defined in relation to the fourth aspect as a hole transporting material and/or an emissive material in an optical device.

In the fifth aspect, preferably the optical device comprises a light-emitting device. More preferably, the light-emitting device comprises an electroluminescent device.

The electroluminescent device many emit light by fluorescence and/or phosphorescence.

When the crosslinked polymer is used as an emissive material, an emissive layer of the device comprises the crosslinked polymer. When an emissive layer of the device comprises the crosslinked polymer, an electron transporting layer may be deposited over the emissive layer from a solution in a solvent. Preferred solution deposition techniques include spin-coating and inkjet printing. Inkjet printing is particularly preferred.

When the crosslinked polymer is used as a hole transporting material, an emissive layer and/or a hole transport layer of the device may comprise the crosslinked polymer.

When a hole transport layer of the device comprises the crosslinked polymer, the emissive layer may be deposited over the hole transport layer from a solution in a solvent. Preferred solution deposition techniques include spin-coating and inkjet printing. Inkjet printing is particularly preferred.

Although its use is not so limited, a hole transport polymer according to this invention and particularly the preferred hole transport polymer described in relation to the fourth aspect of the present invention, advantageously may be used in a hole transport layer of an electroluminescent device, which emits light by phosphorescence. In this case, the hole transport layer should have a wider T₁-T₀ energy gap than the phosphorescent material used in this electroluminescent device. This may be particularly advantageous for green phosphorescence. Quenching of green phosphorescence is a problem with some existing hole transport materials when used in optical devices.

A sixth aspect of the present invention provides a method of making a layer of an optical device comprising the steps of:
(a) providing a plurality of monomers containing at least 1 mol % of crosslinking monomers as defined in relation to the first aspect;
(b) polymerising the plurality of monomers to form a polymer;
(c) depositing the polymer from step (b) on a substrate; and
(d) crosslinking the polymer after deposition to form a crosslinked polymer.

In the method according to the sixth aspect of the present invention, preferably, each of the aryl or heteroaryl groups on which a Y and/or Y¹ reactive leaving group is located in the plurality of crosslinking monomers as defined in relation to the first aspect is conjugatively linked by a direct bond to a double bond or another aryl or heteroaryl group during polymerising in step (b). In this way, it is possible to form a conjugated polymer in step (b).

It is further preferred that the polymer formed in step (b) of the method according to the sixth aspect is a conjugated polymer.

In either case the anode typically will be supported on a glass or plastic substrate. In the embodiment where the crosslinked polymer is to be used as an emissive material in the optical device, typically the substrate in step (c) is a hole transport layer.

A seventh aspect of the present invention provides an optical device containing a layer comprising a crosslinked polymer as defined in relation to the first aspect.

In the seventh aspect, preferably, the optical device comprises a light-emitting device. More preferably, the light-emitting device comprises an electroluminescent device.

In the seventh aspect, typically, the layer comprising the crosslinked polymer is a hole transporting layer. In the case of light-emitting device having an anode, a cathode, and a light emitting layer situated between the anode and the cathode, the hole transporting layer is situated between the anode and the light-emitting layer.

An eighth aspect of the present invention provides a crosslinkable polymer comprising a carbazole repeat unit.

It will be understood that a proportion of the repeat units in the crosslinkable polymer contain a crosslinkable group. Preferably from 1 to 25 mol %, more preferably from 5 to 25 mol % or 1 to 15 mol %, still more preferably from 1 to 10 mol % or 5 to 15 mol %, even more preferably from 5 to 10 mol % of the repeat units in the crosslinkable polymer contain a crosslinkable group. The crosslinkable groups may be pendent from the polymer backbone. The crosslinkable groups may be terminal groups. Suitable crosslinkable groups are as defined anywhere herein.

Some or all of the carbazole repeat units may each contain one or more crosslinkable groups, for example as exemplified in relation to the third embodiment of the first aspect of the present invention. Preferred non-crosslinkable carbazole repeat units comprise general formula 23 defined above.

The crosslinkable polymer may further contain a non-carbazole co-repeat unit. The non-carbazole co-repeat unit may contain one or more crosslinkable groups. Examples of non-carbazole co-repeat units containing one or more crosslinkable groups are triarylamine co-repeat units as defined in relation to the first, second and fourth embodiments of the first aspect of the present invention

The polymer according to the eighth aspect may further contain non-crosslinkable co-repeat units, for example as disclosed anywhere herein.

The polymer according to the eighth aspect may be conjugated. Typically, the polymer according to the eighth aspect will be semiconducting. The polymer according to the eighth aspect may be soluble, preferably in common organic solvents as described herein.

A ninth aspect of the present invention provides a method for making a crosslinkable polymer as defined in relation to the eighth aspect.

The crosslinkable polymer as defined in relation to the eighth aspect may be prepared by polymerisation of suitable monomers. Suitable monomers include monomers comprising a repeat unit of the polymer with reactive leaving groups (Y and Y¹) attached to what would be the termini of the repeat unit in the polymer backbone:

Y-repeat unit-Y¹

Y and Y¹ may be as defined anywhere herein. Suitable polymerisation techniques are as described herein in relation to the third aspect of the present invention.

A tenth aspect of the present invention provides a crosslinked polymer comprising a carbazole repeat unit. The crosslinked polymer according to the tenth aspect may be preparable from a polymer as defined in relation to the eighth aspect. Crosslinking of a polymer as defined in relation to the eighth aspect may be initiated by any suitable means depending on the crosslinkable group. Suitable means include chemical, heat or UV initiation. Typically, during device manufacture, crosslinking is initiated after the crosslinkable polymer as defined in relation to the eighth aspect has been deposited as a film, for example by solution processing.

The crosslinked polymer according to the tenth aspect is envisaged to be useful for hole transport and as a host material in an organic light-emitting device.

An eleventh aspect of the present invention provides an optical device containing a layer comprising a crosslinked polymer according to the tenth aspect. Typically, the layer comprising the crosslinked polymer according to the tenth aspect is a hole transporting layer. The optical device may comprise a light-emitting device. In the case of light-emitting device having an anode, a cathode, and a light emitting layer situated between the anode and the cathode, the hole transporting layer is situated between the anode and the light-emitting layer.

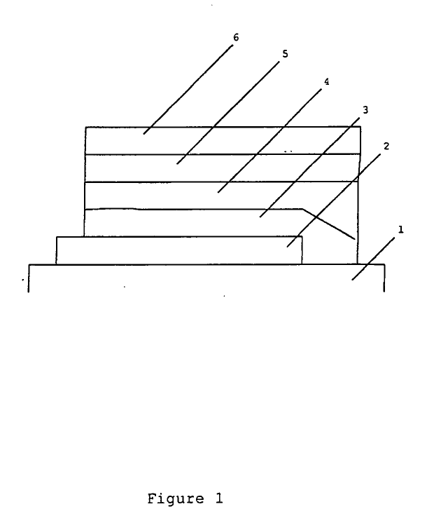

The present invention now will be described in more detail with reference to FIG. 1, which shows a suitable device structure for an optical device according to the seventh and eleventh aspects of the present invention.

### EXAMPLE 1

### (A) Preparation of a Crosslinking Monomer

### i) Monomer N1

Monomer N1 was prepared according to the reaction scheme below:

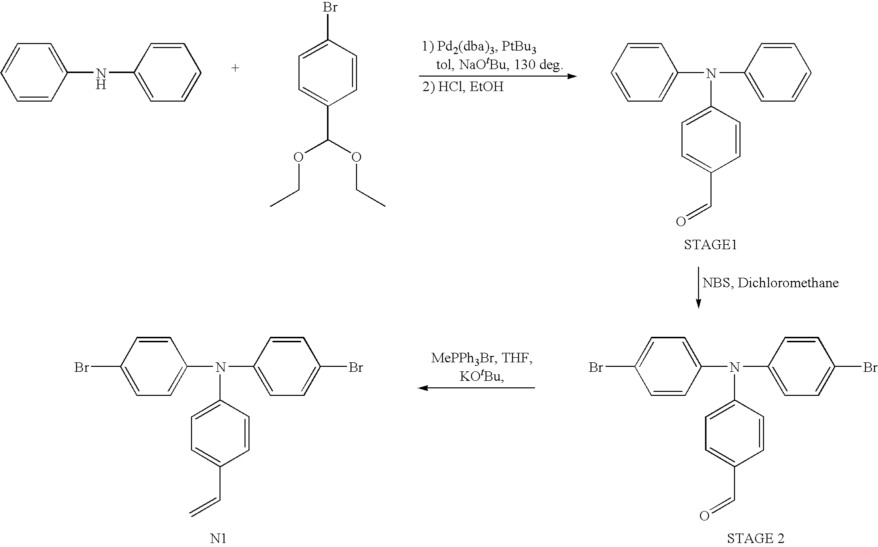

The skilled person will be able to use the above reaction scheme to prepare further crosslinking monomers according to the invention.

### ii) Monomer N2

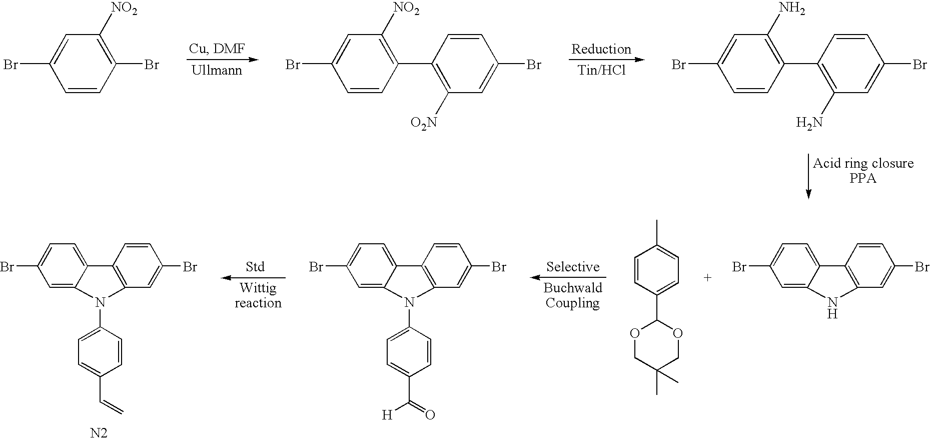

### (B) Preparation of a Polymer

The cross-linkable amine monomer referred to as N1 was polymerised at 5%, 7% and 10% incorporation into polyfluorene type polymers

A general procedure is described below for 7 mole % incorporation of the N1 monomer.

### Generic Polymerisation Procedure

The following monomers were dissolved in 100 ml of toluene, (3.819 g, 7.2 mmol), 9,9′-dioctyl fluorene-2,7-diethyldiboronate (0.996 g 1.8 mmol), bis(4-bromophenyl)-4-sec-butylphenyl amine (3.554 g 7.7 mmol), and bis(4-bromophenyl)-4-vinylphenyl amine (0.5407 g, 1.26 mmol) along with an organo palladium catalyst (0.027 mmol). To the stirring reaction mixture was added 0.0423 mmol of a hydroxide base. The reaction mixture was then stirred, heated to and maintained at reflux for between 12-24 hours, after which the reaction mixtures was sequentially end capped first by adding 1 ml of bromobenzene followed by stirring at reflux, then 1 g of phenyl boronic acid was added and the reaction mixture was again stirred at reflux.

The reaction mixture was then allowed to cool and purified by precipitation into methanol to yield about 4.0 g of polymer which was analysed by GPC in THF using polystyrene standards. The peak molecular weight was about 67,000.

### (C) Preparation of a Light-Emitting Device

Light-emitting devices are made each having a hole transport layer 3 of one of the polymers prepared in (B) above. The polymers are laid down as films using solution processing. The polymer films are crosslinked after deposition by heating in a nitrogen glove box at 200° C. for 1 hour.

The structure of the devices is shown in FIG. 1. The anode 2 is a layer of transparent indium-tin oxide (“ITO”) supported on a glass or plastic substrate 1. The anode 2 layer has a thickness between 1000-2000 Å, usually about 1500 Å. The cathode 5 is a Ca layer having an approximate thickness of 1500 Å. Between the electrodes is a light emissive layer 4 having a thickness up to about 1000 Å. The hole transport layer 3 has a thickness of about 1000 Å. Layer 6 is an encapsulant layer of a suitable thickness.

### COMPARATIVE EXAMPLE

A polymer P1 according to the invention was prepared in accordance with the above method. The polymer P1 is an AB copolymer of alternating fluorene and amine repeat units wherein 5% of the amine units comprise a styryl cross-linking group.

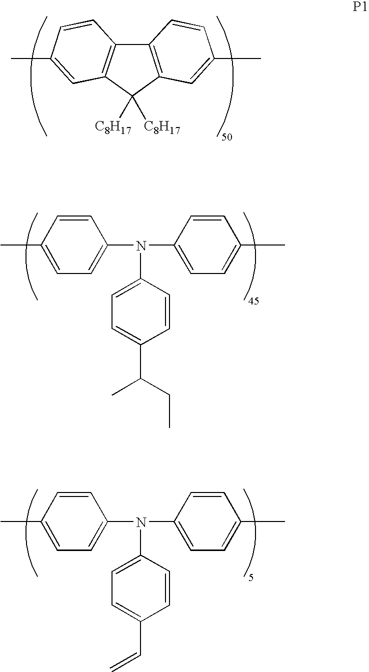

For the purpose of comparison, comparative polymer C1 was prepared in accordance with the above method. The polymer C1 is an AB copolymer of alternating fluorene and amine repeat units wherein 5% of the fluorene units comprise a styryl cross-linking group.

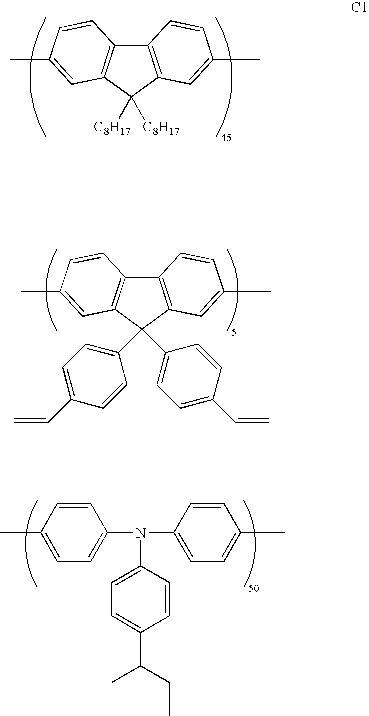

Films of polymer P1 and polymer C1 were deposited by spin-coating from xylene solution onto a glass substrate carrying an ITO anode and a layer of PEDOT. The deposited polymer films were then heated at 180° C. or 200° C. followed by rinsing with xylene. Thickness of each polymer film before and after heating and rinsing was measured.

| | Crosslink | Thickness pre- | Thickness post- | Pre/ |
|---|---|---|---|---|
| | temperature | crosslink & | crosslink & | post |
| Polymer | (° C.) | rinse (nm) | rinse (nm) | ratio |
| P1 | 180 | 75 | 52 | 0.69 |
| C1 | 180 | 63 | 14 | 0.22 |
| P1 | 200 | 75 | 75 | 1 |
| C1 | 200 | 63 | 14 | 0.22 |

As can be seen from the above examples, layers of the crosslinked comparative polymer C1 are significantly thinner following crosslinking and solvent rinsing, indicating that a large proportion of the polymer has not been rendered insoluble. In contrast, the layer of crosslinked polymer P1 according to the invention is much more resistant to solvent indicating a higher degree of crosslinking.

Without wishing to be bound by any theory, the inventors believe that the superior crosslinking of polymer P1 may be attributable to activation of the crosslinking styryl group by the lone pair of electrons on the nitrogen atom:

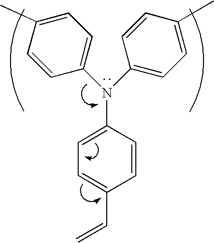

### EXAMPLE 2

### Preparation of a Crosslinking Monomer

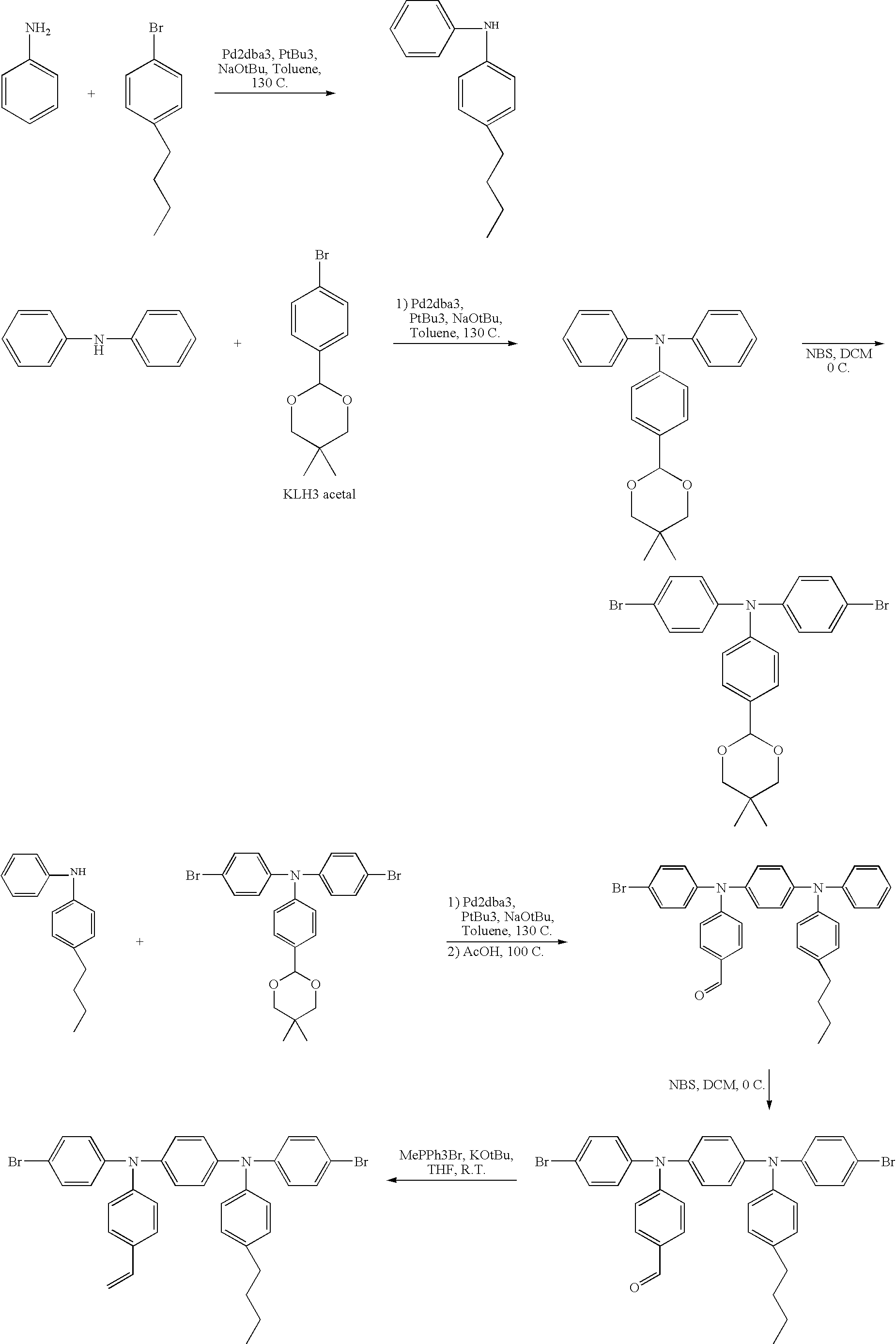

The monomer can be polymerised according to the general polymerisation procedure described in relation to Example 1 (B).

1. A crosslinking monomer for making a crosslinked polymer, said monomer comprising reactive leaving groups Y and Y¹ located on the same or different aryl or heteroaryl groups of the monomer and a structural unit of general formula I: 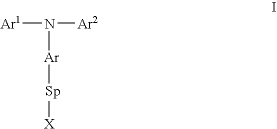
where Ar represents an aryl or heteroaryl group; Ar¹ and Ar² each independently represents a substituted or unsubstituted aryl or heteroaryl group and where any two of Ar, Ar¹ and Ar² may optionally be linked; Sp represents an optional spacer group; and X represents a crosslinkable group.

2. A crosslinking monomer according to claim 1, wherein X represents a styrene group.

3. A crosslinking monomer according to claim 1, wherein the monomer comprises general formula II:

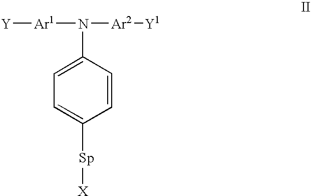

4. A crosslinking monomer according to claim 3, comprising general formula III:

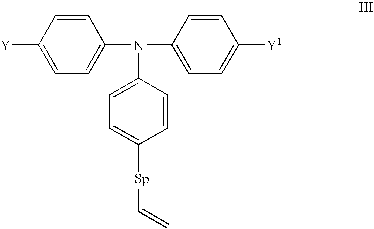

5. A monomer according to claim 1, wherein the monomer comprises general formula VIII:

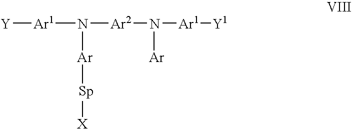

6. A monomer according to claim 1, wherein the monomer comprises general formula XVIII:

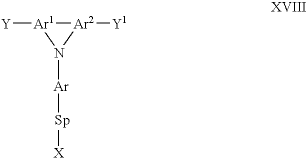

7. A monomer according to claim 1, wherein the monomer comprises general formula XIII:

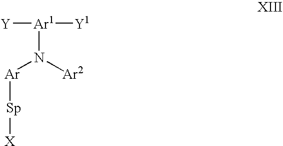

8. A monomer according to claim 1, wherein the monomer comprises general formula XVI: 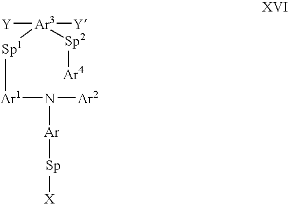
where Sp¹ and Sp² each independently represents an optional spacer group; Ar³ represents an aryl or heteroaryl group; and Ar⁴ represents an aryl or heteroaryl group.

9. A crosslinking monomer according to claim 1, wherein Y and Y¹ independently are selected from the group consisting of boronic acids, boronic acid esters, boranes, halides and triflates.

10. A crosslinking monomer according to claim 9, wherein Y and Y¹ independently are selected from the group consisting of bromine and boronic acid ester.

11. (canceled)

12. A method for producing a crosslinkable polymer comprising the steps of:
(i) providing a plurality of monomers containing at least 1 mol % of crosslinking monomers; and
(ii) polymerizing the plurality of monomers to form a polymer; wherein each crosslinking monomer comprises reactive leaving groups Y and Y¹ located on the same or different aryl or heteroaryl groups of the monomer and a structural unit of general formula I: 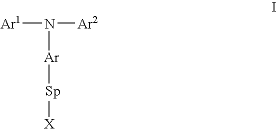
where Ar represents an aryl or heteroaryl group; Ar¹ and Ar² each independently represents a substituted or unsubstituted aryl or heteroaryl group and where any two of Ar, Ar¹ and Ar² may optionally be linked; Sp represents an optional spacer group; and X represents a crosslinkable group.

13. A method according to claim 12, wherein X of each crosslinking monomer represents a styrene group.

14. A method according to claim 12, wherein the plurality of monomers are provided in solution in step (i).

15. A method according to claim 12, wherein the plurality of monomers further contains non-crosslinking monomers.

16. A method according to claim 15, wherein the plurality of monomers further contains a plurality of non-crosslinking monomers, each containing a fluorene group.

17. A method according to claim 16, wherein the fluorene group is 9,9 dioctylfluorene.

18. A method according to claim 16, wherein the plurality of monomers contains from 5 to 25 mol % of crosslinking monomers.

19. A method according to claim 12, wherein the plurality of monomers consists of crosslinking monomers.

20. A method according to claim 12, wherein each aryl or heteroaryl group on which a Y and/or Y¹ reactive leaving group is located in the plurality of crosslinking monomers is conjugatively linked by a direct bond to a carbon-carbon double bond, aryl group or heteroaryl group in another monomer during the polymerizing in step (ii).

21. A method according to claim 12, wherein the method further includes crosslinking the polymer to form a crosslinked polymer.

22. A method according to claim 21, wherein the crosslinking is performed by heating.

23. A crosslinkable polymer preparable by the method according to claim 12.

24. A crosslinkable polymer according to claim 23 comprising a conjugated polymer.

25. (canceled)

26. (canceled)

27. (canceled)

28. A method of making a layer of an optical device comprising the steps of:
(a) providing a plurality of monomers containing at least 1 mol % of crosslinking monomers, where each crosslinking monomer comprises reactive leaving groups Y and Y¹ located on the same or different aryl or heteroaryl groups of the monomer and a structural unit of general formula I: 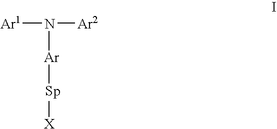
where Ar represents an aryl or heteroaryl group; Ar¹ and Ar² each independently represents a substituted or unsubstituted aryl or heteroaryl group and where any two of Ar, Ar¹ and Ar² may optionally be linked; Sp represents an optional spacer group; and X represents a crosslinkable group;
(b) polymerizing the plurality of monomers to form a polymer;
(c) depositing the polymer from step (b) on a substrate; and
(d) crosslinking the polymer after deposition to form a crosslinked polymer.

29. A method according to claim 28, wherein X of each crosslinking monomer represents a styrene group.

30. A method according to claim 28, wherein the polymer is deposited in solution in step (c).

31. A method according to claim 28, wherein the substrate in step (c) is the anode.

32. An optical device containing a layer comprising a crosslinked polymer preparable by a method according to claim 21.

33. An optical device according to claim 32, wherein the optical device comprises a light-emitting device.

34. An optical device according to claim 33, wherein the light-emitting device comprises an electroluminescent device.

35. An optical device according to claim 32, wherein the layer comprising the crosslinked polymer is a hole transporting layer or an emissive layer.
